(19) United States
(12) Patent Application Publication
(10) Pub. No.: US 2010/0330007 A1
(43) Pub. Date: Dec. 30, 2010

(54) Imrpoved Skin Brightening Compositions

(75) Inventors: Ralph Spindler, Palatine, IL (US); Stepphen J. Urbanec, Arlington Heights, IL (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: AMCOL INTERNATIONAL CORPORATION, Hoffman Estates, IL (US)

(21) Appl. No.: 12/864,167

(22) PCT Filed: Jan. 30, 2009
(86) PCT No.: PCT/US09/32528

§ 371 Date: Sep. 2, 2010

Related U.S. Application Data
(60) Provisional application No. 61/025,546, filed on Feb. 1, 2008.

(51) Int. Cl. A61K 8/67; A61K 8/86; A61K 8/81; A61K 8/97; A61K 8/72; A61Q 17/02; A61Q 15/00; A61Q 17/04; A61Q 19/02; A61P 17/18; A61P 31/10; A61P 31/04; A61P 29/00; A61P 17/00; A61Q 19/00

(52) U.S. Cl. 424/59; 424/62

(57) ABSTRACT

An improved cosmetic and dermatological composition and a method for treating hyperpigmented skin is disclosed. The composition demonstrates an ability to lighten mammalian skin color and is nonirritating. The composition contains (a) a retinoid loaded onto polymeric microparticles and (b) a second skin lightening agent, like kojic acid or a derivative thereof, such as kojic dipalmitate.

### CROSS-REFERENCE TO RELATED APPLICATIONS

This application claims the benefit of U.S. provisional patent application No. 61/025,546, filed Feb. 1, 2008, incorporated herein by reference.

### FIELD OF THE INVENTION

The present invention relates to improved cosmetic and dermatological compositions capable of treating hyperpigmented skin. More particularly, the present invention relates to compositions having an enhanced ability to lighten the color of mammalian skin.

### BACKGROUND OF THE INVENTION

The epidermis of mammals can exhibit hyperpigmentation, e.g., a skin color that is esthetically too dark or uneven in tone. The American Academy of Dermatology has estimated that 5 to 6 million Americans will suffer from a skin pigmentation condition at some point of their lives. It also has been reported that melasma occurs in 50% to 70% of pregnant females, and that about 90% of light-skinned Caucasians over 60 years old develop liver spots. Hyperpigmentation can have a profound negative impact on the social, emotional, and psychological well-being of an individual.

Hyperpigmentation, or an abnormally increased pigmentation or melanin deposition, can be attributed to different etiologies, including local hyperpigmentation from drug use (e.g., calcium antagonists), cyanic melasma, senile melasma, vitiglio, adverse sequelae following sclerotherapy, or postinflammatory or traumatic responses. Other local hyperpigmentations can occur during pregnancy (known as gravidic chloasma), after estro-progestative contraception, by photosensitization, or by postlesional cicatrization.

For example, hyperpigmentation includes age spots, “solar lentigo,” or “liver spots,” mainly resulting from excessive sun exposure, and which are common on the hands, face, forearms; melasma in pregnant women (i.e., “the mask of pregnancy”) or in women taking oral contraceptives; drug-induced or postinflammatory skin darkening; and disease-related skin darkening, such as in Addison's disease. Hyperpigmentation also can result from a cumulative sun exposure throughout life, which leads to age spots or “sun-induced freckles.” Hyperpigmentation further can be attributed to an intrinsic genetic profile, e.g., individuals having melanocytes that secrete melanin in the absence of ultraviolet (UV) exposures.

A highly-pigmented skin also may be considered unesthetic to individuals in various ethnic groups, and who therefore wish to reduce skin color. For example, the first depigmenting cream appeared in Korea decades ago as the result of an esthetic desire of some Asian females to have a pale facial complexion. These initial depigmenting creams contained a mercury compound. Mercurials have since been banned because of their neurotoxicity.

On a global basis, the most widely-treated skin care condition is hyperpigmentation. Treatment of hyperpigmentation can be based on a need to lighten the skin overall to a more esthetically or culturally acceptable tone, or a need to help even skin tone, for example uneven skin tone caused by blemishes, wounds, rashes, and acne scarring, especially on darker skin.

The need for efficacious and safe skin lightening products led to a number of other skin lightening agents being developed and tested for efficacy. Current treatments for hyperpigmentation include using a sunscreen, which prevents tyrosinase activity; melanocyte toxicity due to an application of a compound like hydroquinone; exfoliation; inhibition of melanocyte transfer to keratinocytes using a compound like niacinamide; inhibition of tyrosinase; and combinations of such treatments.

Previously-used skin depigmenting agents include peroxides, such as hydrogen peroxide, zinc peroxide, sodium peroxide, benzoyl peroxide, and the like. However, peroxide activity often is coupled with adverse side effects. Several presently used natural compounds partially inhibit melanin synthesis and/or tyrosinase activity, for example, glucosamines, galactosamines, mannosamines, and some plant extracts, whose action has been correlated to blocking of free radicals which are the true stimulating factors of melanogenesis. Plant extracts also suffer from the disadvantages of instability, a lack of standardized product, and low efficacy. Antioxidants, such as vitamins C and E and esters thereof, also exhibit moderate depigmenting activity, with partial inhibition of melanogenesis. However, such antioxidants typically are not sufficiently efficacious. In addition, to prevent premature browning of the composition, a stabilized and less effective derivative of vitamin C typically is used, e.g, magnesium ascorbyl phosphate and sodium ascorbyl phosphate. Azelaic acid also has been used as a depigmenting agent because it demonstrates a competitive inhibition of tyrosinase and of the DNA synthesis within melanocytes. A number of skin lightening compounds have been partially or completely banned because of toxicity and environmental concerns. For example, mercury, hormonal preparations, and oxidizing agents have been banned as skin-lightening agents.

Tyrosinase inhibitors have become increasingly important in cosmetic and medicinal products in the treatment of hyperpigmentation. A few antimelanogenic reagents, such as monobenzone and hydroquinone, also are clinically useful.

Hydroquinone and its derivatives, such as the monomethyl ether of hydroquinone and arbutin, are the most common depigmenting agents used in topical compositions. Hydroquinone operates by interrupting the production of melanin through inhibition of the tyrosinase enzyme. Hydroquinone is a tyrosinase substrate with antagonist and competitive action on tyrosine. Hydroquinone is highly effective, but there are sufficient concerns relating to the toxicity of hydroquinone such that many countries have banned its use except under the care of a dermatologist. Prescription skin lightening compositions may contain 3% to 5%, by weight, hydroquinone. However, the dosage typically is limited to a concentration of 2%, by weight, because hydroquinone is unstable and irritating, and is cytotoxic to melanocytes, with indications of localized granular hyperpigmentation and formation of elastosis, as well as the occurrence of vitiligo after long-term use. See WO 01/17497, incorporated herein by reference.

Many current treatments contain a derivative of hydroquinone, known as arbutin, that can be found in low concentrations in a variety of extracts including Mitracarpus scaber extract, Uva ursi (bearberry) extract, Morus bombycis (mulberry), Morus alba (white mulberry), and Broussonetia papyrifera (paper mulberry). Arbutin derivatives also are available as a neat compound, such as alpha-arbutin, beta-arbutin, and deoxy-arbutin. These compounds are very expensive and require high concentrations to be effective (e.g., greater than 1 wt. % in formulations), which limit arbutin and derivatives to a minority of the products on the market.

Another tyrosinase inhibitor is kojic acid, which is unstable, exhibits weak mutagenicity, and is a skin sensitizer and irritant. Ferulic acid is a relatively nonefficacious tyrosinase inhibitor. Glabridin (licorice extract) suffers from instability and purity concerns, and a high cost. These tyrosinase inhibitors also suffer from a relatively low efficacy because of a poor skin permeation to the target site of action, i.e., the melanocytes.

Kojic acid is a leading active agent used today for skin lightening. Kojic acid is fungi derived and has disadvantages, such as instability, e.g., undergoes photodegradation over time that reduces efficacy; a tendency to discolor from yellow to brown in formulations over time; mutagenicity and tumor promotion; irritation with sensitization potential; and a provocation of skin contact allergies. Kojic acid has been shown to effectively inhibit melanin production, but suffers from the above serious disadvantages. A derivative of kojic acid, known as kojic acid dipalmitate, has been shown to be stable in formulations, but no conclusive evidence of skin lightening efficacy has been shown.

The present invention is directed to using a combination of (a) a retinoid loaded onto, or entrapped, in a polymeric microparticle delivery system and (b) a second skin lightening agent, like kojic acid or a derivative thereof, e.g., kojic dipalmitate, to provide more effectively lightened skin compared to a current FDA approved over the counter (OTC) composition that contains 2% hydroquinone. Other second skin lightening agents include, for example, arbutin, azelaic acid, and various plant extracts.

### SUMMARY OF THE INVENTION

The present invention is directed to cosmetic and dermatological compositions used in a method of treating hyperpigmentation. More particularly, the present invention is directed to compositions that demonstrate an enhanced ability to lighten skin color through use of a polymeric microparticle delivery system to help reduce the irritation associated with relatively high concentrations of a retinoid. In accordance with the present invention, effective skin lightening is achieved by combining a polymeric microparticle entrapped retinoid with a second skin lightening agent, like kojic acid or a derivative thereof, e.g., kojic acid dipalmitate, in an emulsion composition. The efficacy of a present composition is equivalent to or near equivalent to the industry standard for skin lightening obtained by applying a composition containing hydroquinone.

Therefore, one aspect of the present invention is to provide an emulsified composition comprising (a) a retinoid loaded onto polymeric microparticles and (b) one or more second skin lightening agent.

Another aspect of the present method is to provide a method of treating hyperpigmentation in a mammal, including humans, comprising applying a composition comprising (a) one or more second skin lightening agent and (b) a retinoid loaded onto polymeric microparticles to a skin surface of the mammal. The method is capable of lightening dark skin attributed to age spots or a melasma, for example.

Yet another aspect of the present invention is to provide an emulsified composition containing an (a) an entrapped retinoid and (b) one or more second skin lightening agent, and use of the composition as a skin care product, a topical drug product, or a cosmetic product.

These and other aspects and novel features of the present invention will become apparent from the following detailed description of the preferred embodiments.

### DETAILED DESCRIPTION OF THE PREFERRED EMBODIMENTS

Agents for treating hyperpigmentation often act by inhibiting the biosynthesis of melanins. One example is to inhibit tyrosinase activity, and thereby preclude conversion of tyrosine to melanin. A number of tyrosinase inhibitors are known, and some have been used to treat hyperpigmentation, i.e., to lighten skin. Among the potent tyrosinase inhibitors are the retinoids.

As used herein, the term “hyperpigmentation” is an actual or a perceived skin impairment of excessive dark color. The skin impairment can be actual, i.e., attributed to age, excessive sun exposure, or a disease or condition leading to dark skin areas. The dark skin areas can be in the form of spots, blotches, or relatively large areas of dark color. The skin impairment also can be perceived, i.e., a perception by an individual that his/her skin shade is too dark and the individual has a cosmetic desire to lighten the skin shade.

Accordingly, compositions of the present invention are useful in treating a variety of skin hyperpigmentations, for example, depigmenting melasma, i.e., dark patches of pigmentation on the face and other parts of the body, or for voluntary whitening skin pigmentation.

Typically, dark skin is attributed to an elevated level of melanin. In accordance with the present invention, the composition and method can be used to treat hyperpigmentation, i.e., to lighten dark skin, or to prevent hyperpigmentation, i.e., to reduce or eliminate the production of excessive amounts of melanin and thereby preclude darkening of skin. Therefore, the present invention is directed to a composition comprising (a) a retinoid entrapped in polymeric microparticles and (b) one or more second skin lightening agent, like kojic acid and kojic derivatives, and use of the composition in a method of treating hyperpigmentation.

One active agent in a present composition and method is a retinoid. The retinoid can be, for example, but not limited to, retinol, retinal, retinol acetate, retinol palmitate, retinoic acid, and mixtures thereof. In accordance with the present invention, the retinol is loaded onto polymeric microparticles.

Absorbent polymeric microparticles useful in the present invention have an ability to absorb several times their weight of a liquid compound, such as a retinoid. One preferred class of adsorbent microparticles is prepared by a suspension polymerization technique, as set forth in U.S. Pat. Nos. 5,677,407; 5,712,358; 5,777,054; 5,830,967; 5,834,577, 5,955,552; and 6,107,429, each incorporated herein by reference (available commercially under the tradename of POLY-PORE® E200, INCI name, allyl methacrylate copolymer, from AMCOL International, Hoffman Estates, Ill.). Another preferred class of adsorbent microparticles is prepared by a precipitation polymerization technique, as set forth in U.S. Pat. Nos. 5,830,960; 5,837,790, 6,248,849; and 6,387,995, each incorporated herein by reference (sold under the tradename of POLY-PORE® L200 by AMCOL International, Hoffman Estates, Ill.). These adsorbent microparticles also can be modified after the incorporation of an active compound to modify the rate of release of such a compound, as set forth in U.S. Pat. No. 6,491,953, incorporated herein by reference.

Another useful class of adsorbent polymers prepared by a precipitation polymerization technique is disclosed in U.S. Pat. Nos. 4,962,170; 4,948,818; and 4,962,133, each incorporated herein by reference, and are commercially available under the tradename POLYTRAP® from AMCOL International. Other useful, commercially available adsorbent polymers include, for example, MICROSPONGE® (a copolymer of methyl methacrylate and ethylene glycol dimethacrylate), available from AMCOL International and Poly-HIPE polymers (e.g., a copolymer of 2-ethylhexyl acrylate, styrene, and divinylbenzene) available from Biopore Corporation, Mountain View, Calif.

In particular, the adsorbent polymer microparticles prepared by the suspension polymerization technique, e.g., POLY-PORE® E200, are a highly porous and highly crosslinked polymer in the form of open (i.e., broken) spheres and sphere sections characterized by a mean unit particle size of about 0.5 to about 3,000 microns, preferably about 0.5 to about 300 microns, more preferably about 0.5 to about 100 microns, and most preferably about 0.5 to about 80 microns. A significant portion of the spheres is about 20 microns in diameter.

The polymeric microparticles are oil and water adsorbent, and have an extremely low bulk density of about 0.008 gm/cc to about 0.1 gm/cc, preferably about 0.009 gm/cc to about 0.07 gm/cc, and more preferably about 0.0095 gm/cc to about 0.04-0.05 gm/cc. The microparticles are capable of holding and releasing oleophilic (i.e., oil soluble or dispersible), as well as hydrophilic (i.e., water soluble or dispersible), active agents, individually, or both oleophilic and hydrophilic compounds simultaneously.

The adsorbent polymer microparticles prepared by the suspension polymerization technique include at least two polyunsaturated monomers, preferably allyl methacrylate and an ethylene glycol dimethacrylate, and, optionally, monounsaturated monomers. The microparticles are characterized by being open to their interior, due either to particle fracture upon removal of a porogen after polymerization or to subsequent milling. The microparticles have a mean unit diameter of less than about 50 microns, preferably less than about 25 microns, and have a total adsorption capacity for organic liquids, e.g., mineral oil, that is at least about 72% by weight, preferably at least about 93% by weight, and an adsorption capacity for hydrophilic compounds and aqueous solutions of about 70% to about 89% by weight, preferably about 75% to about 89% by weight, calculated as weight of material adsorbed divided by total weight of material adsorbed plus dry weight of polymer. In a preferred embodiment, the broken sphere microparticles are characterized by a mean unit diameter of about 1 to about 50 microns, more preferably of about 1 to about 25 microns, most preferably, of about 1 to about 20 microns.

Preferred polymeric microparticle delivery systems comprise a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof. Specific polymeric microparticles useful in the present invention can be the previously described POLY-PORE'® E200, POLY-PORE® L200, POLYTRAP® 6603, POLYTRAP® 7603, MICROSPONGE® entrapments, or Poly-HIPE particles, for example.

To function as a delivery system for an active agent, e.g., a retinoid, the active agent is incorporated onto the polymeric microparticles. This can be accomplished by spraying or adding the active agent either directly to the microparticles in such a manner that an essentially homogeneous distribution of the active ingredient is achieved on all the microparticles. Alternatively, if the active agent is a solid compound, the active agent first is dissolved in a suitable volatile solvent, the resulting solution is added to the microparticles, then the volatile solvent is removed under vacuum with optional gentle heating. In some cases, this process is repeated several times to achieve a desired loading level of the active agent. Another method of loading a solid active agent that is not readily soluble in a volatile solvent is to first disperse the solid in a suitable carrier, such as polyether or polyol, and then add the dispersion directly to the polymeric microparticles.

The load of the active agent in and on the polymeric microparticles can be about 1 to about 80 wt. %, when the active ingredient is a solid material at room temperature (i.e., about 23° C. to 25° C.), or in a preferred amount of about 5 to about 67 wt. %, or in the more preferred amount of about 10 to about 50 wt. %, by total weight of the loaded microparticles.

In some cases, it is possible to further protect the active agent by adding a second material, usually a liquid or waxy material, to the loaded microparticles after the active agent has been entrapped. This is especially effective for reactive active agents. After entrapping the active agent, a barrier layer (i.e., a secondary entrapment), optionally, can be applied to the loaded microparticles to prevent rapid diffusion of the active agent from the microparticle. Also, the melting point of the barrier layer can be selected such that it melts at a temperature higher than the highest temperature that the microparticles will be exposed either during storage in the final formulated product or during accelerated aging of the final formulation.

Examples of materials that can be used as a barrier layer or a secondary entrapment, include, but are not limited to, low melting alcohols (C₈ through C₂₀) and fatty alcohols ethoxylated with one to three moles of ethylene oxide. Examples of fatty alcohols and alkoxylated fatty alcohols include, but are not limited to, behenyl alcohol, caprylic alcohol, cetyl alcohol, cetaryl alcohol, decyl alcohol, lauryl alcohol, isocetyl alcohol, myristyl alcohol, oleyl alcohol, stearyl alcohol, tallow alcohol, stearety-2, ceteth-1, cetearth-3, and laureth-2. Additional fatty alcohols and alkoxylated alcohols are listed in the International Cosmetic Ingredient Dictionary and Handbook, Tenth Edition, Volume 3, pages 2127 and pages 2067-2073 (2006), incorporated herein by reference.

Another class of materials that can be used as a barrier layer is the C₈ to C₁₂ fatty acids, including, but not limited to, stearic acid, capric acid, behenic acid, caprylic acid, lauric acid, myristic acid, tallow acid, oleic acid, palmitic acid, isostearic acid and additional fatty acids listed in the International Cosmetic Ingredient Dictionary and Handbook, Tenth Edition, Volume 3, page 2126-2127, incorporated herein by reference. The barrier material also can be a hydrocarbon, like mineral oil, 1-decene dimer, polydecene, paraffin, petrolatum, vegetable-derived petrolatum or isoparafin.

Another class of barrier materials is waxes, like mink wax, carnauba wax, and candelilla wax, for example, and synthetic waxes, like silicone waxes, polyethylene, and polypropylene. Fats and oils can be useful barrier material agents, which include, for example, but are not limited to, lanolin oil, linseed oil, coconut oil, olive oil, menhaden oil, castor oil, soybean oil, tall oil, rapeseed oil, palm oil, and neatsfoot oil, and additional fats and oils listed in the International Cosmetic Ingredient Dictionary and Handbook, Tenth Edition, Volume 3, pages 2124-2126. Another useful class of barrier materials includes water-insoluble esters having at least 10 carbon atoms, and preferable 10 to about 32 carbon atoms. Numerous esters are listed in International Cosmetic Ingredient Dictionary and Handbook, Tenth Edition, Volume 3, pages 2115-2123.

Alternatively, an active agent can be mixed with a molten waxy material, then entrapped in a microparticle delivery system. In the case of liquid active agents, the waxy materials disclosed above as barrier materials also can be used as an additive for thickening the liquid active agent and thereby help minimize premature diffusion of the active agent from the microparticle.

The amount of the waxy material can be about 10% to about 67%, based on the weight of the loaded microparticles. In a more preferred embodiment, the amount of the waxy material is about 25% to about 50 wt. %, based on the total weight of the loaded microparticles.

Retinoids that can be loaded onto, or entrapped by, the polymeric microparticles include, both naturally occurring and synthetic compounds having the general structure of vitamin A (retinol) and variations of that structure having similar biological and pharmacological activity as retinol. Examples of retinoids include, but are not limited to, retinol, retinal, retinyl acetate, retinyl palmitate, retinoic acid, retinyl propionate, retinyl linoleate, dehydroretinol, eretinate, eretrin, motretinide, and mixtures thereof. U.S. Pat. No. 5,851,538, incorporated herein by reference, discloses several additional useful retinoids.

Polymeric microparticle delivery systems (e.g., U.S. Pat. No. 5,851,538, incorporated herein by reference) have been shown to improve the stability and reduce the irritation associated with the use of retinoids and their analogs, including retinol. Both formulation stability and skin irritation are problems often associated with using these highly effective compounds, especially at the relatively high concentrations of retinol that provide the greatest efficacy in skin lightening.

U.S. Pat. No. 6,896,890, incorporated herein by reference, discloses that an oil-in-water emulsion containing hydroquinone entrapped in a microparticle delivery system in addition to an entrapment of retinol in a microparticle delivery system, together with free hydroquinone, demonstrates improved stability compared to a control emulsion that contained only free hydroquinone.

Another component of a present composition is one or more second skin lightening agent, for example, kojic acid, a derivative thereof, and mixtures thereof. The one or more skin lightening agent is present in the free form, i.e., is not loaded onto the polymeric microparticles The one or more second skin lightening agent present in the composition in an amount of about 0.1% to about 5%, by weight, of the composition; preferably about 0.25% to about 3%, by weight; and more preferably about 0.5% to about 2%, by weight.

In addition to kojic acid and derivatives thereof, the one or more second skin whitening agent can be azelaic acid, hydroquinone, the monobenzyl or monomethyl ether of hydroquinone, mequinol (4-hydroxyanisole), glabridin (licorice extract), magnesium ascorbyl phosphate, extracts of Arctostaphylos patula and/or Arctostaphylos viscida, extract of Emblica officinalis, glucosamines, galactosamines, mannosamines, Mitracarpus scaber extract, Uva ursi (bearberry) extract, Morus bombycis (mulberry) extract, Morus alba (white mulberry) extract, and Broussonetia papyrifera (paper mulberry) extract, arbutin including alpha-arbutin, beta-arbutin, and deoxy-arbutin, sodium ascorbyl phosphate, a peroxide, like hydrogen peroxide, zinc peroxide, sodium peroxide, or benzoyl peroxide, cysteine, 4-thioresorcin, 3-aminotyrosine, 5-hydroxy-2-hydroxymeth-γ-pyridone, fomesjaponicus and ganoderma extracts, glycyrrhizinic acid, hydroquinone-β-glucoside, catharanthus roseus extract, proteoglycans, proteinase inhibitors, oligopeptides, betaines, methyl 4-benzyloxy-2-hydroxybenzoate, and 4-benzyloxy-2-hydroxybenzoic acid, and mixtures thereof.

Especially preferred second skin lightening agents are kojic acid (5-hydroxy-4-pyran-4-one-2-methyl) and aliphatic esters of kojic acid, as disclosed in U.S. Pat. No. 4,369,174, incorporated herein by reference. The kojic acid esters have a structural formula:

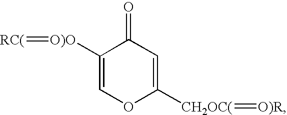

wherein each R, independently, is an aliphatic group containing 2 to 19 carbon atoms. Each R group also is optionally is substituted, independently, with one or more, e.g., one to three, OH and/or CO₂H group. The kojic acid esters disclosed in U.S. Pat. No. 4,369,174 are prepared from kojic acid and a C₃-C₂₀ aliphatic carboxylic acid, and preferably a C₈-C₂₀ aliphatic carboxylic acid. The aliphatic carboxylic acid can be saturated or unsaturated, a dicarboxylic acid, or a hydroxycarboxylic acid. Preferred kojic acid esters are kojic acid dipalmitate, kojic acid distearate, kojic acid dioleate, kojic acid dimyristate, and kojic acid dicaprylate. Especially preferred is kojic acid dipalimitate.

### Example 1

The following oil-in-water emulsion containing a combination of entrapped retinol loaded onto polymeric microparticles and free unentrapped kojic acid dipalmitate was prepared as follows:

| | Ingredients | Wt. % |
|---|---|---|
| Phase A | 1 Deionized Water | 63.83 |
| | 2 Magnesium Aluminum Silicate | 0.5 |
| | 3 Glycerin | 3 |
| | 4 Na₂EDTA | 0.2 |
| | 5 Triethanolamine | 0.6 |
| | 6 Methylparaben | 0.2 |
| Phase B | 7 Cetyl Ricinoleate | 2 |
| | 8 Cetyl Alcohol | 2 |
| | 9 C10-30 Cholesterol/Lanosterol Esters | 2.5 |
| | 10 Emulsifing Wax NF | 4.5 |
| | 11 Dimethicone (350 cst) | 2 |
| | 12 Vitamin E Acetate | 0.3 |
| | 13 PEG-10 Soy Sterol | 0.5 |
| | 14 Stearic Acid | 1 |
| | 15 Ascorbyl Phosphate | 0.05 |
| | 16 BHT | 0.2 |
| | 17 Cyclomethicone | 1 |
| | 18 Caprylic/Capric Triglyceride | 6 |
| | 19 Cetyl Phosphate | 1 |
| | 20 PEG-100 Stearate | 2.5 |
| | 21 Kojic Dipalmitate | 1.3 |
| | 22 Alpha bisabolol | 0.1 |
| Phase C | 23 Microsponge Retinol Entrapment | 0.72 |
| | (contains about 23.5% retinol) | |
| | 24 Deionized Water | 2.5 |
| | 25 Benzyl Alcohol | 1 |
| | 26 Phenoxyethanol | 0.5 |

Manufacturing Procedure: Admix Phase A components and heat to 75° C. Combine Phase B ingredients and heat to 75° C. Add Phase B to Phase A and homogenize until a uniform emulsion has been formed. Cool the emulsion to below 45° C. and add in Phase C with homogenizing, if needed. Cool to ambient temperature and package the emulsion.

### Example 2

The following oil-in-water emulsion containing a combination of entrapped retinol loaded onto polymeric microparticles and free unentrapped alpha arbutin and licorice extract was prepared as follows:

| | Ingredients | Wt % |
|---|---|---|
| Phase A | Water | 53.09 |
| | Magnesium Aluminum Silicate | 0.5 |
| | Na₂EDTA | 0.2 |
| | NaOH (50 wt. %) | 0.1 |
| | Glycerin | 3 |
| | Cetearyl Glycoside | 0.5 |
| Phase B | Cetyl Ricinoleate | 2 |
| | Glyceryl Stearate | 2 |
| | Cetyl Alcohol | 3 |
| | Cetyl Phosphate | 1.5 |
| | Dimethicone, 350 cst | 2.5 |
| | Shea Butter | 0.5 |
| | Stearic Acid | 0.5 |
| | Vitamin E Acetate | 0.1 |
| | BHT | 0.1 |
| | Cyclomethicone | 1.5 |
| | Caprylic/Capric Triglyceride | 8 |
| Phase C | Water | 5 |
| | Microsponge Retinol Entrapment | 0.8 |
| | (contains 23.5% retinol) | |
| | Alpha Arbutin | 1 |
| | Water | 10 |
| | Butylene Glycol | 3 |
| | Licorice extract | 0.01 |
| | alpha biabolol | 0.1 |
| | Euxyl 9010 (Schulke Mayr) | 1 |

Manufacturing Procedure: Mix Phase A ingredients together at 80° to 85° C. Mix Phase B ingredients together at 80° to 85° C. Add Phase B to Phase A with mixing then homogenize the phases using a high shear mixer to form an emulsion. Cool the emulsion to less than 45° C., then add the components of Phase C, being careful to protect the product from light and oxygen.

### Example 3

The irritation potential for the emulsion of Example 1 was compared to a commercial retinol cream that did not contain a second skin brightening agent (ROC Retinol Correxion Deep Wrinkle Cream). The commercial cream contains 0.11%, by weight, retinol, whereas the formulation of Example 1 contains 0.17%, by weight, retinol, as analyzed by HPLC. A cumulative irritation study (N=20) was conducted via a side by side analysis of the two compositions. Each composition (0.2 g) was placed on a hypoallergenic occlusive patch and each panelist wore the patch for 24 hours. At the end of 24 hours, the patch was removed by a trained technician and skin irritation, as judged by a five point scale, was determined. This process was repeated for a total of 14 days. The cumulative score was determined by adding each of the individual scores for each of the panelists. The T-Test was used to determine the P score using 95% confidence level. The results summarized in the table below show that even though the composition in Example 1 contains 54% more retinol than the commercial product, the irritation score for the composition of Example 1 is 70% lower, which is statistically significant.

| | Cumulative | |
|---|---|---|
| | Irritation | |
| Product | Score | p (<0.05) |
| Example 1 (0.17% retinol) | 57 | 0.0311 |
| ROC Retinol Correxion Deep | 80.5 | |
| Wrinkle Night Cream (0.11%) | | |

### Example 4

To measure the efficacy of the composition of Example 1, a clinical study was performed wherein ten panelists applied the composition of Example 1 one half of their face. To the other side of their face, each applied a commercial hydroquinone containing product that contains 2% hydroquinone (Porcelana Fade Dark Spots Nighttime Treatment). This application was repeated twice a day, in the morning and in the evening. After the morning application of the composition, a sunscreen (SPF 55) was applied to the skin to minimize the effect of the sun on the skin during the day. Photographs of the panelists were taken at time=0, 4, 8 and 13 weeks using regular light, polarized light, and UVA light. The photographic results were judged by two expert graders. Each product was found to be efficacious by both expert graders, as well as by self assessment. With all three lighting techniques, the graders found that there was some improvement on both sides of the face on each assessment day, but there was no difference in the degree of improvement between the side of the face treated with Example 1 and the side treated with the commercial product. Therefore, the product of Example 1 performs similarly to that of the commercial product.

The panelists in the study also assessed the performance of the two products. In this phase of the study, 70% to 80% of the panelists found that the emulsion of Example 1 faded age spots, while 50% to 70% of the panelists found that the control product faded age spots. Although no statistical difference existed between the results, the results show that the panelists felt that the two products provided at least similar efficacy.

A present composition also can contain an optional second active skin-lightening agent. Useful second active skin-lightening agents include, but are not limited to, skin exfoliants; hydroquinone or a derivative thereof, such as benzylhydroquinone ether; ascorbic acid or a derivative thereof, such as magnesium ascorbyl phosphate; a caffeic acid or ester thereof; a benzofuran, such as 5- or 6-hydroxybenzofuran; a plant extract, such as licorice, mulberry, heather, and angelica ashitaba; a pearl extract; a steroidal antiinflammatory agent of the hydrocortisone-type and the like; a nonsteroidal antiinflammatory agent selected from the group consisting of acetylsalicylic acid, acetaminophen, naproxen, and fenamic acid derivatives, such as the sodium salt; an antiinflammatory agent, such as alpha-bisabolol, beta-glycyrrhetinic acid, allantoin, aloe extract, rosmarinic acid, azulene or a derivative thereof, asiaticoside, sericoside, ruscogenin, escin, escolin, quercetin, rutin, betulinic acid or a derivative thereof, catechin or a derivative thereof; and mixtures thereof.

A present composition is useful in personal care, cosmetic, and pharmaceutical compositions. The present compositions provide an effective delivery of a retinoid to lighten the skin without irritation. The resulting compositions for skin lightening can be formulated with other topically applied active agents, in addition to, or in lieu of, an optional second active skin-lightening agent to achieve both skin lightening and a second cosmetic or therapeutic effect different from skin lightening.

In accordance with an important feature of the present invention, a topically applied compound for providing a second cosmetic or therapeutic effect can be any of a wide variety of compounds, either water soluble or oil soluble.

Such a topically applied active compound, therefore, can be one of, or a mixture of, a cosmetic compound, a medicinally active compound, a compound used in cosmetics or personal care, or any other compound that is useful upon topical application to the skin. Such topically active agents include, but are not limited to, skin-care compounds, plant extracts, antioxidants, insect repellants, counterirritants, vitamins, steroids, antibacterial compounds, antifungal compounds, antiinflammatory compounds, topical anesthetics, sunscreens, optical brighteners, and other cosmetic and medicinal topically effective compounds.

For example, a skin conditioner can be the topically applied compound. Skin conditioning agents include, but are not limited to, humectants, such a fructose, glucose, glycerin, propylene glycol, glycereth-26, mannitol, urea, pyrrolidone carboxylic acid, hydrolyzed lecithin, coco-betaine, cysteine hydrochloride, glucamine, PPG-15, sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamins A, E, and D, amino-functional silicones, ethoxylated glycerin, alpha-hydroxy acids and salts thereof, fatty oil derivatives, such as PEG-24 hydrogenated lanolin, and mixtures thereof. Numerous other skin conditioners are listed in the CTFA Cosmetic Ingredient Handbook, First Ed., J. Nikotakis, ed., The Cosmetic, Toiletry and Fragrance Association (1988), (hereafter CTFA Handbook), pages 79-84, incorporated herein by reference.

The skin conditioner also can be a water-insoluble ester having at least 10 carbon atoms, and preferably 10 to about 32 carbon atoms. Suitable esters include those comprising an aliphatic alcohol having about eight to about twenty carbon atoms and an aliphatic or aromatic carboxylic acid including from two to about twelve carbon atoms, or conversely, an aliphatic alcohol having two to about twelve carbon atoms with an aliphatic or aromatic carboxylic acid including about eight to about twenty carbon atoms. The ester is either straight-chained or branched. Suitable esters, therefore, include, for example, but are not limited to:
(a) aliphatic monohydric alcohol esters, including, but not limited to:
myristyl propionate,
isopropyl isostearate,
isopropyl myristate,
isopropyl palmitate,
cetyl acetate,
cetyl propionate,
cetyl stearate,
isodecyl neopentanoate,
cetyl octanoate,
isocetyl stearate;
(b) aliphatic di- and tri-esters of polycarboxylic acid, including, but not limited to:
diisopropyl adipate,
diisostearyl fumarate,
dioctyl adipate, and
triisostearyl citrate;
(c) aliphatic polyhydric alcohol esters, including, but not limited to:
propylene glycol dipelargonate;
(d) aliphatic esters of aromatic acids, including, but not limited to:
C₁₂-C₁₅ alcohol esters of benzoic acid,
octyl salicylate,
sucrose benzoate, and
dioctyl phthalate.
   Numerous other esters are listed in the CTFA Handbook, at pages 24 through 26, incorporated herein by reference.

The topically applied compound also can be an antioxidant or an optical brightener, like a distyrylbiphenyl derivative, stilbene or a stilbene derivative, a pyralozine derivative, or a coumarin derivative. Optical brighteners useful as the topically applied compound can be any compound capable of absorbing an invisible UV portion of the daylight spectrum, and converting this energy into the longer visible wavelength portion of the spectrum. The optical brightener is colorless on the substrate, and does not absorb energy in the visible part of the spectrum. The optical brightener typically is a derivative of stilbene or 4,4′-diaminostilbene, biphenyl, a 5-membered heterocycle, e.g., triazole, oxazole, or imidazole, or a 6-membered heterocycle, e.g., a coumarin, a naphthalamide, or an s-triazine.

The optical brighteners are available under a variety of tradenames, such as TINOPAL®, LEUCOPHOR®, and CALCOFLUOR®. Specific fluorescent compounds include, but are not limited to, TINOPAL® 5BM, CALCOFLUOR® CG, and LEUCOPHOR® BSB.

In addition, other compounds can be included in a present composition as the topically active compound in an amount sufficient to perform their intended function. For example, sunscreen compounds such as benzophenone-3, tannic acid, uric acids, quinine salts, dihydroxy naphtholic acid, an anthranilate, p-aminobenzoic acid, phenylbenzimidazole sulfonic acid, PEG-25, or p-aminobenzoic acid can be used as the topically applied compound. Further, sunscreen compounds such as dioxybenzone, ethyl 4-[bis(hydroxypropyl)]aminobenzoate, glyceryl aminobenzoate, homosalate, methyl anthranilate, octocrylene, octyl methoxycinnamate, octyl salicylate, oxybenzone, padimate O, red petrolatum, titanium dioxide, 4-menthylbenzylidene camphor, benzophenone-1, benzophenone-2, benzophenone-6, benzophenone-12, isopropyl dibenzoyl methane, butyl methoxydibenzoylmethane, zotocrylene, or zinc oxide can be used as the topically applied compound. Other sunscreen compounds are listed in CTFA Handbook, pages 86 and 87, incorporated herein by reference.

Similarly, topically applied drugs, like antifungal compounds, antibacterial compounds, antiinflammatory compounds, topical anesthetics, skin rash, skin disease, and dermatitis medications, and antiitch and irritation-reducing compounds can be used as the active agent in the compositions of the present invention. For example, analgesics such as benzocaine, dyclonine hydrochloride, aloe vera, and the like; anesthetics such as butamben picrate, lidocaine hydrochloride, xylocalne, and the like; antibacterials and antiseptics, such as povidone-iodine, polymyxin b sulfate-bacitracin, zinc-neomycin sulfate-hydrocortisone, chloramphenicol, ethylbenzethonium chloride, erythromycin, and the like; antiparasitics, such as lindane; essentially all dermatologicals, like acne preparations, such as benzoyl peroxide, erythromycin benzoyl peroxide, clindamycin phosphate, 5,7-dichloro-8-hydroxyquinoline, and the like; antiinflammatory agents, such as alclometasone dipropionate, betamethasone valerate, and the like; burn relief ointments, such as o-amino-p-toluenesulfonamide monoacetate, and the like; dermatitis relief agents, such as the active steroid amcinonide, diflorasone diacetate, hydrocortisone, and the like; diaper rash relief agents, such as methylbenzethonium chloride, and the like; emollients and moisturizers, such as mineral oil, PEG-4 dilaurate, lanolin oil, petrolatum, mineral wax, and the like; fungicides, such as butocouazole nitrate, haloprogin, clotrimazole, and the like; herpes treatment drugs, such as O-[(2-hydroxymethyl)-methyl]guanine; pruritic medications, such as alclometasone dipropionate, betamethasone valerate, isopropyl myristate MSD, and the like; psoriasis, seborrhea, and scabicide agents, such as anthralin, methoxsalen, coal tar, and the like; steroids, such as 2-(acetyloxy)-9-fluoro-1′,2′,3′,4′-tetrahydro-11-hydroxypregna-1,4-dieno-[16,17-b]naphthalene-3,20-dione and 21-chloro-9-fluoro-1′,2′,3′,4′-tetrahydro-11b-hydroxypregna-1,4-dieno-[16,17-b]naphthalene-3,20-dione. Any other medication capable of topical administration, like skin protectants, such as allantoin, and antiacne agents, such as salicylic acid, also can be incorporated in a composition of the present invention in an amount sufficient to perform its intended function. Other topically applied compounds are listed in Remington's Pharmaceutical Sciences, 17th Ed., Mack Publishing Co., Easton, Pa. (1985), pages 773-791 and pages 1054-1058 (hereinafter Remington's), incorporated herein by reference.

The topically active compound also can be a plant extract or a natural oil. Nonlimiting plant extracts are those obtained from alfalfa, aloe vera, amla fruit, angelica root, anise seed, apple, apricot, artichoke leaf, asparagus root, banana, barberry, barley sprout, bee pollen, beet leaf, bilberry fruit, birch leaf, bitter melon, black currant leaf, black pepper, black walnut, blueberry, blackberry, burdock, carrot, cayenne, celery seed, cherry, chickwood, cola nut, corn silk, cranberry, dandelion root, elderberry, eucalyptus leaf, flax oil powder, ginger root, gingko leaf, ginseng, goldenrod, goldenseal, grape, grapefruit, guava, hibiscus, juniper, kiwi, kudzu, lemon, licorice root, lime, malt, marigold, myrrh, olive leaf, orange fruit, orange peel, oregano, papaya fruit, papaya leaf, passion fruit, peach, pear, pine bark, plum, pomegranate, prune, raspberry, rhubarb root, rosemary leaf, sage leaf, spearmint leaf, St. John's wart, strawberry, sweet cloves, tangerine, violet herb, watercress, watermelon, willow bark, wintergreen leaf, witch hazel bark, yohimbe, and yucca root. An example of a natural oil is rice bran oil.

The present compositions can be admixed with other ingredients traditionally included in cosmetic, dermatological, medicinal, and other such compositions. These ingredients include, but are not limited to, dyes, fragrances, preservatives, antioxidants, detackifying agents, and similar types of compounds. The ingredients are included in the composition in an amount sufficient to perform their intended function.

The following additional ingredients typically are included in a present composition. Each of these ingredients, and any other ingredient, is present in a sufficient amount to perform its intended function, without adversely affecting the efficacy of the retinoid-loaded microparticles and the kojic acid and/or kojic acid derivatives with respect to treating hyperpigmentation.

For example, a present composition can contain a surfactant. The surfactant can be an anionic surfactant, a cationic surfactant, a nonionic surfactant, or a compatible mixture of surfactants. The surfactant also can be an ampholytic or amphoteric surfactant, which have anionic or cationic properties depending upon the pH of the composition.

A present composition also can contain a hydrotrope. A hydrotrope is a compound that has an ability to enhance the water solubility of other compounds. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, sodium camphor sulfonate, and disodium succinate.

A present composition also can contain an additional organic solvent. The solvent can be a water-soluble organic compound containing one to six, and typically one to three, hydroxyl groups, e.g., alcohols, diols, triols, and polyols. Specific examples of solvents include, but are not limited to, methanol, ethanol, isopropyl alcohol, n-butanol, n-propyl alcohol, ethylene glycol, propylene glycol, glycerol, diethylene glycol, dipropylene glycol, tripropylene glycol, hexylene glycol, butylene glycol, 1,2,6-hexanetriol, sorbitol, PEG-4,1,5-pentanediol, similar hydroxyl-containing compounds, and mixtures thereof. The solvent also can be water or an aprotic solvent, e.g., dimethyl sulfoxide or tetrahydrofuran.

A present composition also can contain a thickening or gelling agent. A thickening or gelling agent can be, for example, a polymer that is water soluble or that generates a colloidal solution in water. A thickening or gelling agent, therefore, can be, for example, polymers or copolymers unsaturated carboxylic acids or unsaturated esters, polysaccharide derivatives, gums, colloidal silicates, polyethylene glycols (PEG) and their derivatives, polyvinylpyrrolidones and their derivatives, polyacrylamides and their derivatives, polyacrylonitriles, hydrophilic silica gels, or mixtures thereof.

Specific thickening or gelling agents can be, for example, acrylic and/or methacrylic polymers or copolymers, vinylcarboxylic polymers, polyglyceryl acrylates or methacrylates, polyacrylamides derivatives, cellulose or starch derivatives, chitin derivatives, alginates, hyaluronic acid and its salts, chonodroit n sulphates, xanthan, gellan, Rhamsan, karaya or guar gum, carob flour, and colloidal aluminum magnesium silicates of the montmorillonite type.

Additional thickening or gelling agents include vinylcarboxylic polymers sold under the tradename CARBOPOL® (Goodrich), acrylic acid/ethyl acrylate copolymers, acrylic acid/stearyl methacrylate copolymers, carboxymethylcellulose, hydroxymethylcellulose, hydroxypropylcellulose, microcrystalline cellulose, hydroxypropyl guar, colloidal hectorites, bentonites, and the like.

Other classes of optional ingredients included in a present composition can be, but not limited to, pH adjusters, chelating agents, preservatives, buffering agents, foam stabilizers, opacifiers, and similar classes of ingredients known to persons skilled in the art. Specific optional ingredients include inorganic phosphates, sulfates, and carbonates as buffering agents; EDTA and phosphates as chelating agents; and acids and bases as pH adjusters.

Nonlimiting examples of basic pH adjusters are ammonia; mono-, di-, and tri-alkyl amines; mono-, di-, and tri-alkanolamines; alkali metal and alkaline earth metal hydroxides; and mixtures thereof. Specific, nonlimiting examples of basic pH adjusters are ammonia; sodium, potassium, and lithium hydroxide; monoethanolamine; triethylamine; isopropanolamine; diethanolamine; and triethanolamine. Examples of acidic pH adjusters are the mineral acids and organic carboxylic acids. Nonlimiting examples of mineral acids are citric acid, hydrochloric acid, nitric acid, phosphoric acid, and sulfuric acid.

In the personal care area, the present compositions can be designed as cosmetic basecoats and undercoats, bath capsules, bath oils, bath tablets, bath salts, bath soaps, blushers, face, body, and hand creams and lotions, cosmetic foundations, hormone creams and lotions, leg and body paints, makeup bases, makeup fixatives, makeup products, moisturizing creams and lotions, night creams and lotions, paste masks, skin care products, skin fresheners, skin lighteners, tonics, dressings, and wrinkle smoothing creams and lotions.

In particular, a present composition can be a lotion; makeup preparation, like makeup foundations; skin care preparation, like hand lotion, vanishing cream, night cream, sunscreen, body lotion, facial cream, clay mask, moisturizing lotion, make-up remover, antiacne preparation, antiaging preparation, and sebum control; analgesic and cortisonal steroid creams and preparations; insect repellants; and facial masks and revitalizers. The compositions also can be incorporated into plasters, bandages, dressings, gauze pads, and similar articles.

A composition of the present invention is topically applied to the skin as needed in order to lighten skin color to a desired degree. Typically, the composition is topically applied to the skin one to four times per day. However, application of a present composition can be more or less frequent as prescribed, required, or desired. The present compositions are applied to the skin by spraying or rubbing. The preferred route of administration is rubbing onto the skin with a soft massage to ensure intimate contact with the skin.

Obviously, many modifications and variations of the invention as hereinbefore set forth can be made without departing from the spirit and scope thereof and, therefore, only such limitations should be imposed as are indicated by the appended claims.

What is claimed is:

1. A skin-lightening composition comprising a retinoid loaded onto polymeric microparticles and a second skin lightening agent.

2. The composition of claim 1 wherein the polymeric microparticles are highly crosslinked and are derived from methacrylate monomers, acrylate monomers, or mixtures thereof.

3. The composition of claim 1 wherein the polymeric microparticles comprise an allyl methacrylate copolymer, an ethylene glycol dimethacrylate/allyl methacrylate copolymer, a lauryl methacrylate/ethylene glycol dimethacrylate copolymer, and mixtures thereof.

4. The composition of claim 1 wherein the polymeric microparticles are selected from the group consisting of a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

5. The composition of claim 1 wherein the polymeric microparticles comprise a copolymer of allyl methacrylate and ethylene glycol dimethacrylate, a copolymer of ethylene glycol dimethacrylate and lauryl methacrylate, or a mixture thereof.

6. The composition of claim 5 wherein the polymeric microparticles comprise copolymer of ethylene glycol dimethacrylate and methyl methacrylate.

7. The composition of claim 1 wherein the second skin lightening agent is selected from the group consisting of azelaic acid, hydroquinone, monobenzyl ether of hydroquinone, monomethyl ether of hydroquinone, azelaic acid, mequinol, glabridin, licorice extract, magnesium ascorbyl phosphate, extract of Arctostaphylos patula, extract of Arctostaphylos viscida, extract of Emblica officinalis, a glucosamine, a galactosamine, a mannosamine, Mitracarpus scaber extract, Uva ursi extract, Morus bombycis extract, Morus alba extract, and Broussonetia papyrifera extract, arbutin, alpha-arbutin, beta-arbutin, deoxy-arbutin, sodium ascorbyl phosphate, a peroxide, hydrogen peroxide, zinc peroxide, sodium peroxide, benzoyl peroxide, cysteine, 4-thioresorcin, 3-aminotyrosine, 5-hydroxy-2-hydroxymeth-γ-pyridone, fomesjaponicus extract, ganoderma extract, glycyrrhizinic acid, hydroquinone-β-glucoside, catharanthus roseus extract, a proteoglycan, a proteinase inhibitor, an oligopeptide, a betaine, methyl 4-benzyloxy-2-hydroxybenzoate, 4-benzyloxy-2-hydroxybenzoic acid, and mixtures thereof.

8. The composition of claim 1 where the second skin lightening agent is selected from the group consisting of kojic acid, arbutin, derivatives thereof, and mixtures thereof.

9. The composition of claim 8 wherein the skin lightening agent comprises kojic acid, a kojic acid ester, or a mixture thereof.

10. The composition of claim 9 wherein the kojic acid ester has a structure 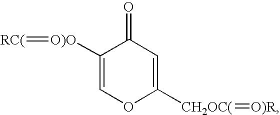
wherein each R, independently, is an aliphatic group containing 2 to 19 carbon atoms, and each R is optionally and independently substituted with one or more OH and/or CO₂H group.

11. The composition of claim 8 wherein the kojic acid ester is selected from the group consisting of kojic acid distearate, kojic acid dipalmitate, kojic acid dioleate, kojic acid dimyristate, kojic acid dicaprylate, and mixtures thereof.

12. The composition of claim 1 where the second skin lightening agent comprises kojic acid dipalmitate.

13. The composition of claim 1 wherein the retinoid is selected form the group consisting of retinol, retinal, retinyl acetate, retinyl palmitate, retinoic acid, retinyl propionate, retinyl linoleate, dehydroretinol, eretinate, eretrin, motretinide, and mixtures thereof.

14. The composition of claim 1 further comprising a topically applied compound selected from the group consisting of a pesticide, a drug, a therapeutic agent, a deodorant, a skin conditioner, an antioxidant, an insect repellant, a counterirritant, a vitamin, a plant extract, a steroid, an antibacterial compound, an antifungal compound, an antiinflammatory compound, a topical anesthetic, an epidermal lipid replacement, a sunscreen, an optical brightener, a dermatitis or skin disease medication, and mixtures thereof.

15. A method of treating mammalian skin comprising a step of contacting the skin with a composition of claim 1 in a sufficient amount to lighten skin color.

16. The method of claim 15 wherein the mammalian skin is human skin.

17. The method of claim 16 wherein the mammalian skin is hyperpigmented.

18. The method of claim 17 wherein the hyperpigmentation is attributed to drug use, cyanic melasma, senile melasma, vitiglio, adverse sequelae following sclerotherapy, a postinflammatory response, a posttraumatic response, pregnancy, estro-progestative contraception, excessive sun exposure, photosensitization, postlesional cicatrization, Addisons disease, or an intrinsic genetic profile.

19. A method of preventing hyperpigmented mammalian skin comprising a step of contacting the skin with a composition of claim 1 in a sufficient amount to prevent excessive darkening of the skin.
